(19) United States
(12) Patent Application Publication
(10) Pub. No.: US 2025/0144409 A1
(43) Pub. Date: May 8, 2025

(54) BURR HOLE PLUGS WITH LEAD RETENTION ARRANGEMENTS AND METHODS OF MAKING AND USING

(71) Applicant: Boston Scientific Neuromodulation Corporation, Valencia, CA (US)

(72) Inventor: Zdzislaw Bernard Malinowski, Castaic, CA (US)

(21) Appl. No.: 18/935,291

(22) Filed: Nov. 1, 2024

Related U.S. Application Data
(60) Provisional application No. 63/547,827, filed on Nov. 8, 2023.

(51) Int. Cl. A61N 1/05

(52) U.S. Cl. CPC A61N 1/0539

(57) ABSTRACT

A burr hole plug includes a plug base defining a plurality of grooves and a burr hole aperture for insertion of at least one lead (or multiple leads) through the plug base, wherein each of the grooves is configured for receiving a portion of a lead, wherein each of the grooves is defined by at least one groove wall, wherein, for each of the grooves, the plug base further includes at least one grip extending into the groove from the at least one groove wall to engage, and facilitate retention of, the portion of any lead received within the groove; and a cover configured to be disposed over, and removably coupled to, the plug base for lead retention. Alternatively or additionally, a burr hole plug can include one or more of a solid filler disposed in the burr hole aperture or a clip ring for lead retention.

### CROSS-REFERENCE TO RELATED APPLICATIONS

This application claims the benefit under 35 U.S.C. § 119 (e) of U.S. Provisional Patent Application Ser. No. 63/547,827, filed Nov. 8, 2023, which is incorporated herein by reference.

### FIELD

The present disclosure is directed to the area of implantable stimulation systems and methods of making and using the systems. The present disclosure is also directed to burr hole plugs and implantable electrical stimulation systems including the burr hole plugs, as well as methods of making and using the burr hole plugs and stimulation systems.

### BACKGROUND

Implantable electrical stimulation systems have proven therapeutic in a variety of diseases and disorders. For example, stimulation of the brain, such as deep brain stimulation, can be used to treat a variety of diseases or disorders, and spinal cord stimulation systems have been used as a therapeutic modality for the treatment of chronic pain syndromes. Peripheral nerve stimulation has been used to treat incontinence, as well as a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the brain, nerves, or other tissue to be stimulated. The pulse generator in the control module generates electrical pulses that are delivered by the electrodes to body tissue.

### BRIEF SUMMARY

One aspect is a burr hole plug that includes a plug base defining a plurality of grooves and a burr hole aperture for insertion of at least one lead (or multiple leads) through the plug base, wherein each of the grooves is configured for receiving a portion of a one of the at least one lead, wherein each of the grooves is defined by at least one groove wall, wherein, for each of the grooves, the plug base further includes at least one grip extending into the groove from the at least one groove wall to engage, and facilitate retention of, the portion of any of the at least one lead received within the groove; and a cover configured to be disposed over, and removably coupled to, the plug base for lead retention.

In at least some aspects, for at least one of the grooves, the at least one grip includes two opposing grips extending from the at least one groove wall. In at least some aspects, the grips include polyether ether ketone or polycarbonate.

In at least some aspects, the cover includes a cover base and a plurality of prongs extending from the cover base, wherein the prongs are configured to removably engage the grooves to facilitate retention of the cover on the plug base. In at least some aspects, at least a portion of each of the prongs has a shape complementary to a shape of a portion of at least one of the grooves.

In at least some aspects, the grips of the plug base are configured to compress the portion of the lead when the portion of the lead is received within the groove.

Another aspect is a kit that includes any of the burr hole plugs described above and a lead implantable within the patient through the burr hole plug, wherein the burr hole plug is configured to receive a portion of the lead within a one of the grooves.

A further aspect is a burr hole plug that includes a plug base defining a burr hole aperture for insertion of a lead (or multiple leads) through the plug base; a solid filler filling a portion of the burr hole aperture, wherein the solid filler is configured for insertion of the lead through the solid filler using an insertion tool and configured to fill a gap formed by withdrawal of the insertion tool leaving the lead inserted through the plug base; and a cover configured to be disposed over, and coupled to, the plug base for lead retention.

In at least some aspects, the solid filler includes silicone. In at least some aspects, the solid filler fills a portion of the burr hole aperture that is configured to reside below an outer surface of a skull of a patient when the burr hole plug is implanted into the skull.

In at least some aspects, after the lead is inserted through the filler and the insertion tool is withdrawn, the solid filler is configured to resist at least one of lateral movement, longitudinal movement, or angular movement of a portion of the lead residing within the solid filler. In at least some aspects, the plug base defines a plurality of grooves, wherein each of the grooves is configured for receiving a portion of the lead, wherein each of the grooves is defined by at least one groove wall, wherein, for each of the grooves, the plug base further includes at least one grip extending into the groove from the at least one groove wall to engage, and facilitate retention of, the portion of the lead received within the groove.

Yet another aspect is a burr hole plug that includes a plug base defining a burr hole aperture for insertion of a lead (or multiple leads) through the plug base; a clip ring configured for insertion into the burr hole aperture, the clip ring including a ring portion and a clip portion extending radially inward from the ring portion, wherein the clip portion includes opposing arms and, when a diameter of the ring portion is reduced by application of force, the clip portion is configured to increase a separation distance between at least some portions of the opposing arms to receive a portion of the lead between the opposing arms, wherein, when the force is removed, the ring portion is biased toward a larger diameter, complementary to a diameter of the burr hole aperture, and to engaging and compressing the portion of the lead received by the clip portion of the clip ring to retain the lead; and a cover configured to be disposed over, and coupled to, the plug base for lead retention.

In at least some aspects, the opposing arms include ridges disposed thereon and configured to engage the portion of the lead. In at least some aspects, the ring portion includes two opposing, separated, circumferential portions extending from the clip portion. In at least some aspects, the plug base includes a plurality of ridges arranged around at least a portion of the burr hole aperture to hinder rotation of the clip ring within the burr hole aperture.

In at least some aspects, the burr hole plug includes a plurality of the clip rings, wherein each of the clip rings is configured for receiving a portion of a different lead. In at least some aspects, the plug base defines a plurality of grooves, wherein each of the grooves is configured for receiving a portion of the lead, wherein each of the grooves is defined by at least one groove wall, wherein, for each of the grooves, the plug base further includes at least one grip extending into the groove from the at least one groove wall to engage, and facilitate retention of, the portion of the lead received within the groove.

Another aspect is a kit that includes any of the burr hole plugs described above and the lead implantable within the patient through the burr hole plug.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system;

FIG. 2 is a schematic side view of one embodiment of an electrical stimulation lead;

FIG. 3 is a schematic overview of one embodiment of components of a stimulation system, including an electronic subassembly disposed within a control module;

FIG. 4A is an exploded perspective view of components of one embodiment of a burr hole plug;

FIG. 4B is a top perspective view of the assembled burr hole plug of FIG. 4A;

FIG. 4C is a side view of the assembled burr hole plug of FIG. 4A;

FIG. 4D is a bottom perspective view of a cover of the burr hole plug of FIG. 4A;

FIG. 4E is a top perspective view of a plug base of the burr hole plug of FIG. 4A with leads extending through the plug base;

FIG. 4F is a top perspective view of the assembled burr hole plug of FIG. 4A disposed on a skull of a patient with leads extending out of the burr hole plug;

FIG. 4G is a top perspective view of the plug base of the burr hole plug of FIG. 4A with an inset to show detail of grooves and grips of the plug base;

FIG. 4H is a close-up perspective view of a portion of the plug base of the burr hole plug of FIG. 4A;

FIG. 5A is an exploded perspective view of components of another embodiment of a burr hole plug;

FIG. 5B is a close-up perspective view of a portion of the plug base and solid filler of the burr hole plug of FIG. 5A with a lead and insertion tool inserted through the solid filler;

FIG. 5C is a close-up perspective view of a portion of the plug base and solid filler of the burr hole plug of FIG. 5A with the lead and solid filler after removal of the insertion tool;

FIG. 6A is an exploded perspective view of components of a third embodiment of a burr hole plug;

FIG. 6B is a top perspective view of the plug base and clip rings of the burr hole plug of FIG. 6A;

FIG. 6C is a top perspective view of the assembled burr hole plug of FIG. 6A disposed on a skull of a patient prior to attachment of the cover and with leads extending out of the burr hole plug; and

FIG. 6D is a top perspective view of the assembled burr hole plug of FIG. 6A disposed on a skull of a patient with attached cover and leads extending out of the burr hole plug.

### DETAILED DESCRIPTION

The present disclosure is directed to the area of implantable stimulation systems and methods of making and using the systems. The present disclosure is also directed to burr hole plugs and implantable electrical stimulation systems including the burr hole plugs, as well as methods of making and using the burr hole plugs and stimulation systems.

Suitable implantable electrical stimulation systems include, but are not limited to, a least one lead with one or more electrodes disposed on a distal portion of the lead and one or more terminals disposed on one or more proximal portions of the lead. Leads include, for example, percutaneous leads, paddle leads, cuff leads, or any other arrangement of electrodes on a lead. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734;7,761,165; 7,783,359; 7,792,590; 7,809,446; 7,949,395; 7,974,706; 8,175,710; 8,224,450; 8,271,094; 8,295,944; 8,364,278; 8,391,985; and 8,688,235; and U.S. Patent Applications Publication Nos. 2007/0150036; 2009/0187222; 2009/0276021; 2010/0076535; 2010/0268298; 2011/0005069; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; and 2013/0197602, all of which are incorporated by reference.

In the discussion below, electrical stimulation leads and electrical stimulation systems are exemplified, but it will be understood that optical stimulation leads, optical/electrical stimulation leads, and other leads can be used, as well as optical stimulation systems and optical/electrical stimulation systems. Examples of optical stimulation or modulation systems or electrical/optical stimulation systems, which include one or more optical emitters in addition to, or as an alternative to, electrodes, are found in U.S. Pat. Nos. 9,415,154; 10,335,607; 10,625,072; and 10,814,140 and U.S. Patent Application Publications Nos. 2013/0317572; 2013/0317573; 2017/0259078; 2017/0225007; 2018/0110971; 2018/0369606; 2018/0369607; 2019/0209849; 2019/0209834; 2020/0094047; 2020/0155584; 2020/0376262; 2021/0008388; 2021/0008389; 2021/0016111; and 2022/0072329, all of which are incorporated by reference in their entireties.

A percutaneous lead for electrical stimulation (for example, deep brain stimulation) includes stimulation electrodes that can be ring electrodes, segmented electrodes that extend only partially around the circumference of the lead, or any other type of electrode, or any combination thereof. The segmented electrodes can be provided in sets of electrodes, with each set having electrodes circumferentially distributed about the lead at a particular longitudinal position. A set of segmented electrodes can include any suitable number of electrodes including, for example, two, three, four, or more electrodes. For illustrative purposes, the leads are described herein relative to use for deep brain stimulation, but it will be understood that such leads can be used for applications other than deep brain stimulation, including spinal cord stimulation, peripheral nerve stimulation, dorsal root ganglion stimulation, sacral nerve stimulation, or stimulation of other nerves, muscles, and tissues.

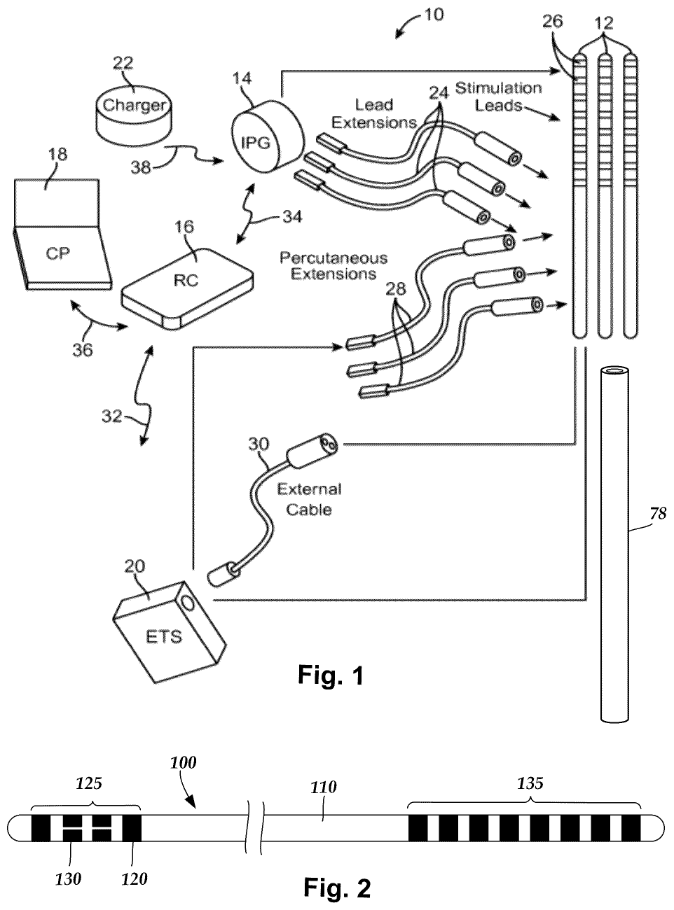

Turning to FIG. 1, one embodiment of an electrical stimulation system 10 includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22. The stimulation lead(s) 12 can be implanted into the patient using an insertion tool 78, such as a cannula, needle, or the like.

The IPG 14 is physically connected, optionally, via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The implantable pulse generator can be implanted into a patient's body, for example, below the patient's clavicle area or within the patient's abdominal cavity. The implantable pulse generator can have eight stimulation channels which may be independently programmable to control the magnitude of the current stimulus from each channel. In some embodiments, the implantable pulse generator can have more or fewer than eight stimulation channels (e.g., 4-, 6-, 16-, 32-, or more stimulation channels). The implantable pulse generator can have one, two, three, four, or more connector ports, for receiving the terminals of the leads and/or lead extensions.

The ETS 20 may also be physically connected, optionally via the percutaneous lead extensions 28 and external cable 30, to the stimulation leads 12. The ETS 20, which may have similar pulse generation circuitry as the IPG 14, also delivers electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a uni- or bi-directional wireless communications link 32. Once the IPG 14 and neurostimulation leads 12 are implanted, the RC 16 may be used to telemetrically communicate with or control the IPG 14 via a uni- or bi-directional communications link 34. Such communication or control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. The CP 18 allows a user, such as a clinician, the ability to program stimulation parameters for the IPG 14 and ETS 20 in the operating room and in follow-up sessions. Alternately, or additionally, stimulation parameters can be programed via wireless communications (e.g., Bluetooth) between the RC 16 (or external device such as a hand-held electronic device) and the IPG 14.

The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters can be subsequently modified by operation of the RC 16 in a stand-alone mode (i.e., without the assistance of the CP 18).

For purposes of brevity, the details of the RC 16, CP 18, ETS 20, and external charger 22 will not be further described herein. Details of exemplary embodiments of these devices are disclosed in U.S. Pat. No. 6,895,280, which is expressly incorporated herein by reference. Other examples of electrical stimulation systems can be found at U.S. Pat. Nos. 6,181,969; 6,516,227; 6,609,029; 6,609,032; 6,741,892; 7,949,395; 7,244,150; 7,672,734; and 7,761,165; 7,974,706; 8,175,710; 8,224,450; and 8,364,278; and U.S. Patent Application Publication No. 2007/0150036, as well as the other references cited above, all of which are incorporated by reference.

Turning to FIG. 2, one or more leads are configured for coupling with a control module. The term “control module” is used herein to describe a pulse generator (e.g., the IPG 14 or the ETS 20 of FIG. 1). Stimulation signals generated by the control module are emitted by electrodes of the lead(s) to stimulate patient tissue. The electrodes of the lead(s) are electrically coupled to terminals of the lead(s) that, in turn, are electrically coupleable with the control module. In some embodiments, the lead(s) couple(s) directly with the control module. In other embodiments, one or more intermediary devices (e.g., a lead extension, an adaptor, a splitter, or the like) are disposed between the lead(s) and the control module.

The leads described herein include 8 electrodes. It will be understood that the leads could include any suitable number of electrodes. The leads can include ring electrodes, a distal-tip electrode, or one or more segmented electrodes, or any combination thereof. Additionally, the term “elongated member” used herein includes leads, as well as intermediary devices (e.g., lead extensions, adaptors, splitters, or the like).

FIG. 2 illustrates one embodiment of a lead 100 (corresponding to lead 12 of FIG. 1) with electrodes 125 disposed at least partially about a circumference of the lead 100 along a distal end portion of the lead and terminals 135 disposed along a proximal end portion of the lead. The lead 100 can be implanted near or within the desired portion of the body to be stimulated such as, for example, the brain. In one example of operation for deep brain stimulation, access to the desired position in the brain can be accomplished by drilling a hole in the patient's skull or cranium with a cranial drill (commonly referred to as a burr), and coagulating and incising the dura mater, or brain covering. The lead 100 can be inserted into the cranium and brain tissue with the assistance of a stylet (not shown). The lead 100 can be guided to the target location within the brain using, for example, a stereotactic frame and a microdrive motor system. In some embodiments, the microdrive motor system can be fully or partially automatic. The microdrive motor system may be configured to perform one or more of the following actions (alone or in combination): insert the lead 100, advance the lead 100, retract the lead 100, or rotate the lead 100.

The lead 100 for deep brain stimulation can include stimulation electrodes, recording electrodes, or both. In at least some embodiments, the lead 100 is rotatable so that the stimulation electrodes can be aligned with the target neurons after the neurons have been located using the recording electrodes.

Stimulation electrodes may be disposed on the circumference of the lead 100 to stimulate the target neurons. Stimulation electrodes may be ring-shaped so that current projects from each electrode equally in every direction from the position of the electrode along a length of the lead 100. In the embodiment of FIG. 2, two of the electrodes 125 are ring electrodes 120. Ring electrodes 120 typically do not enable stimulus current to be directed from only a limited angular range around of the lead. Segmented electrodes 130, however, can be used to direct stimulus current to a selected angular range around the lead 100. When segmented electrodes 130 are used in conjunction with an implantable pulse generator that delivers constant current stimulus, current steering can be achieved to more precisely deliver the stimulus to a position around an axis of the lead (i.e., radial positioning around the axis of the lead 100). To achieve current steering, segmented electrodes 130 can be utilized in addition to, or as an alternative to, ring electrodes 120.

As described above, the lead 100 includes a lead body 110, terminals 135, and one or more ring electrodes 120 and one or more sets of segmented electrodes 130 (or any other combination of electrodes). The lead body 100 can be formed of a biocompatible, non-conducting material such as, for example, a polymeric material. Suitable polymeric materials include, but are not limited to, silicone, polyurethane, polyurea, polyurethane-urea, polyethylene, or the like. Once implanted in the body, the lead 100 may be in contact with body tissue for extended periods of time. In at least some embodiments, the lead 100 has a cross-sectional diameter of no more than 1.5 mm and may be in the range of 0.5 to 1.5 mm. In at least some embodiments, the lead 100 has a length of at least 10 cm and the length of the lead 100 may be in the range of 10 to 70 cm.

The electrodes 125 can be made using a metal, alloy, conductive oxide, or any other suitable conductive biocompatible material. Examples of suitable materials include, but are not limited to, platinum, platinum iridium alloy, iridium, titanium, tungsten, palladium, palladium rhodium, or the like. Preferably, the electrodes are made of a material that is biocompatible and does not substantially corrode under expected operating conditions in the operating environment for the expected duration of use.

Each of the electrodes can either be used (ON) or unused (OFF). When the electrode is used, the electrode can be used as an anode or cathode and carry anodic or cathodic current. In some instances, an electrode might be an anode for a period of time and a cathode for a period of time.

As described above, deep brain stimulation leads and other leads may include one or more sets of segmented electrodes. Segmented electrodes may provide for superior current steering than ring electrodes because target structures in deep brain stimulation are not typically symmetric about the axis of the distal electrode array. Instead, a target may be located on one side of a plane running through the axis of the lead. Through the use of a radially segmented electrode array (“RSEA”), current steering can be performed not only along a length of the lead but also around a circumference of the lead. This provides precise three-dimensional targeting and delivery of the current stimulus to neural target tissue, while potentially avoiding stimulation of other tissue. Examples of leads with segmented electrodes include U.S. Pat. Nos. 8,473,061; 8,571,665; and 8,792,993; U.S. Patent Application Publications Nos. 2010/0268298; 2011/0005069; 2011/0130803; 2011/0130816; 2011/0130817; 2011/0130818; 2011/0078900; 2011/0238129; 2012/0016378; 2012/0046710; 2012/0071949; 2012/0165911; 2012/197375; 2012/0203316; 2012/0203320; 2012/0203321; 2013/0197424; 2013/0197602; 2014/0039587; 2014/0353001; 2014/0358208; 2014/0358209; 2014/0358210; 2015/0045864; 2015/0066120; 2015/0018915; 2015/0051681; U.S. patents applications Ser. Nos. 14/557,211 and 14/286,797; and U.S. Provisional Patent Application Ser. No. 62/113,291, all of which are incorporated herein by reference. Segmented electrodes can also be used for other stimulation techniques including, but not limited to, spinal cord stimulation, peripheral nerve stimulation, dorsal root ganglion stimulation, or stimulation of other nerves, muscles, and tissues.

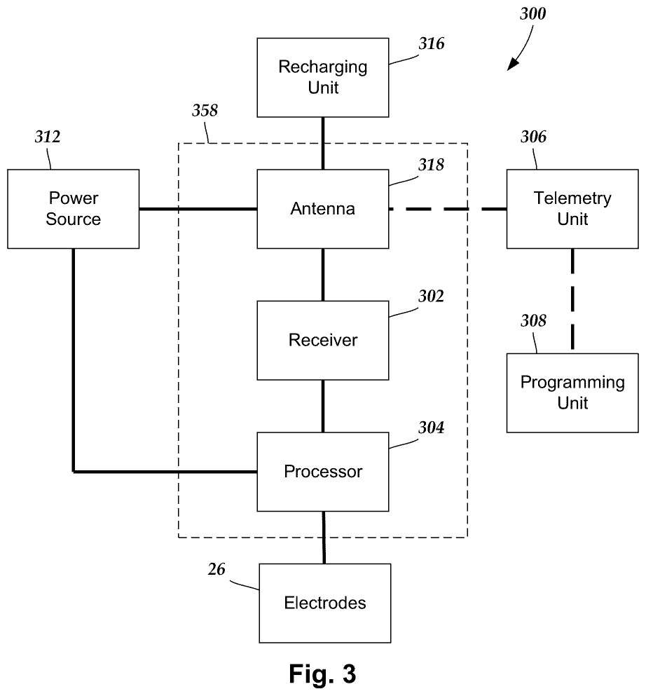

FIG. 3 is a schematic overview of one embodiment of components of an electrical stimulation system 300 including an electronic subassembly 358 disposed within a control module. The electronic subassembly 358 may include one or more components of the IPG. It will be understood that the electrical stimulation system can include more, fewer, or different components and can have a variety of different configurations including those configurations disclosed in the stimulator references cited herein.

Some of the components (for example, a power source 312, one or more antennas 318, a receiver 302, and a processor 304) of the electrical stimulation system can be positioned on one or more circuit boards or similar carriers within a sealed electronics housing of an implantable pulse generator (see e.g., 14 in FIG. 1), if desired. Any power source 312 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 318 or a secondary antenna. In at least some embodiments, the antenna 318 (or the secondary antenna) is implemented using the auxiliary electrically-conductive conductor. The external power source can be in a device that is mounted on the skin of the user or in a unit that is provided near the user on a permanent or periodic basis.

If the power source 312 is a rechargeable battery, the battery may be recharged using the optional antenna 318, if desired. Power can be provided to the battery for recharging by inductively coupling the battery through the antenna to a recharging unit 316 external to the user. Examples of such arrangements can be found in the references identified above. The electronic subassembly 358 and, optionally, the power source 312 can be disposed within a control module (e.g., the IPG 14 or the ETS 20 of FIG. 1).

In one embodiment, electrical stimulation signals are emitted by the electrodes (e.g., electrode array 26 in FIG. 1) to stimulate neural tissue near the electrodes. The processor 304 is generally included to control the timing and electrical characteristics of the electrical stimulation system. For example, the processor 304 can, if desired, control one or more of the timing, frequency, strength, duration, and waveform of the pulses. In addition, the processor 304 can select which electrodes can be used to provide stimulation, if desired. In some embodiments, the processor 304 selects which electrode(s) are cathodes and which electrode(s) are anodes. In some embodiments, the processor 304 is used to identify which electrodes provide the most useful stimulation of the desired tissue.

Various processors can be used and may be an electronic device that, for example, produces pulses at a regular interval or the processor can be capable of receiving and interpreting instructions from an external programming unit 308 that, for example, allows modification of pulse characteristics. In the illustrated embodiment, the processor 304 is coupled to a receiver 302 which, in turn, is coupled to the optional antenna 318. This allows the processor 304 to receive instructions from an external source to, for example, direct the pulse characteristics and the selection of electrodes, if desired.

In one embodiment, the antenna 318 is capable of receiving signals (e.g., RF signals) from an external telemetry unit 306 which is programmed by the programming unit 308. The programming unit 308 can be external to, or part of, the telemetry unit 306. The telemetry unit 306 can be a device that is worn on the skin of the user or can be carried by the user and can have a form similar to a pager, cellular phone, or remote control, if desired. As another alternative, the telemetry unit 306 may not be worn or carried by the user but may only be available at a home station or at a clinician's office. The programming unit 308 can be any unit that can provide information to the telemetry unit 306 for transmission to the electrical stimulation system 300. The programming unit 308 can be part of the telemetry unit 306 or can provide signals or information to the telemetry unit 306 via a wireless or wired connection. One example of a suitable programming unit 308 is a computer operated by the user or clinician to send signals to the telemetry unit 306.

The signals sent to the processor 304 via the antenna 318 and the receiver 302 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 300 to cease operation, to start operation, to start charging the battery, or to stop charging the battery. In other embodiments, the stimulation system does not include the antenna 318 or receiver 302 and the processor 304 operates as programmed.

Optionally, the electrical stimulation system 300 may include a transmitter (not shown) coupled to the processor 304 and the antenna 318 for transmitting signals back to the telemetry unit 306 or another unit capable of receiving the signals. For example, the electrical stimulation system 300 may transmit signals indicating whether the electrical stimulation system 300 is operating properly or not or indicating when the battery needs to be charged or the level of charge remaining in the battery. The processor 304 may also be capable of transmitting information about the pulse characteristics so that a user or clinician can determine or verify the characteristics.

In at least some instances of stimulation of the brain, when a lead is implanted into the brain of a patient, the lead is inserted through a burr hole in the skull of the patient. The lead extends out of the burr hole and is coupled to a control module implanted elsewhere, for example, in the torso of the patient. A burr hole plug is provided in the burr hole to cover the opening through the skull, to protect the lead exiting the skull, and to firmly hold the lead in place to prevent or reduce lead migration within the brain.

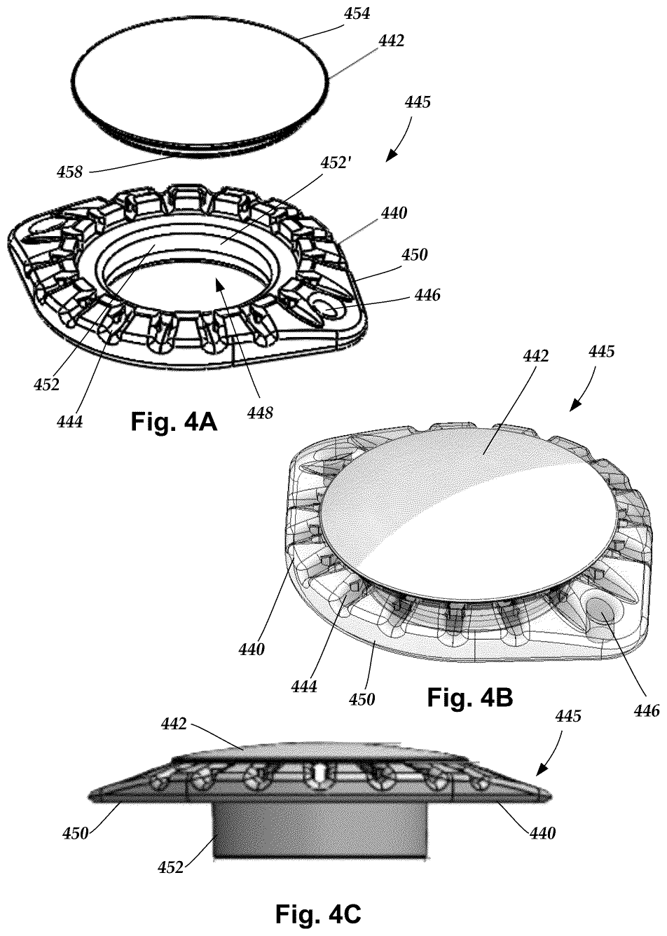

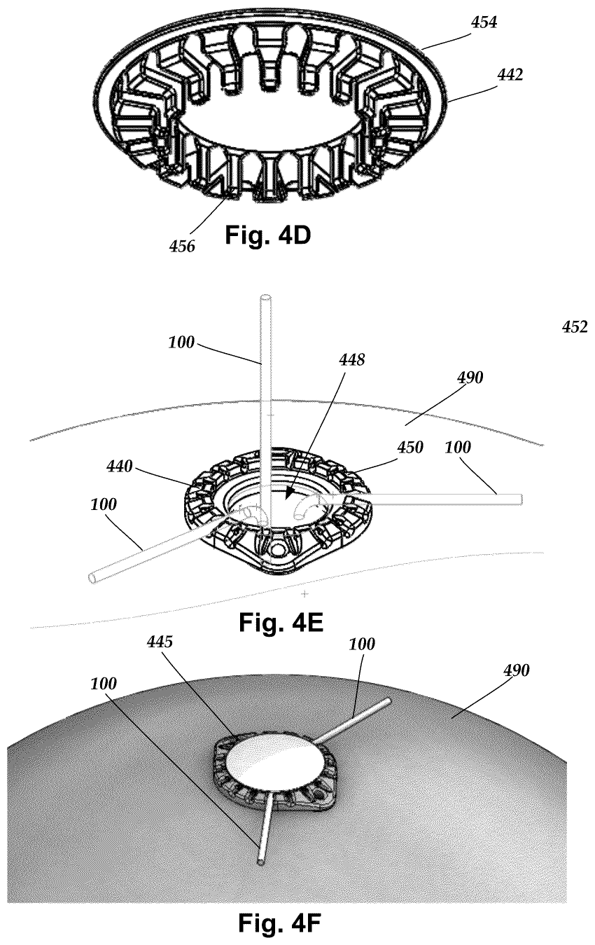

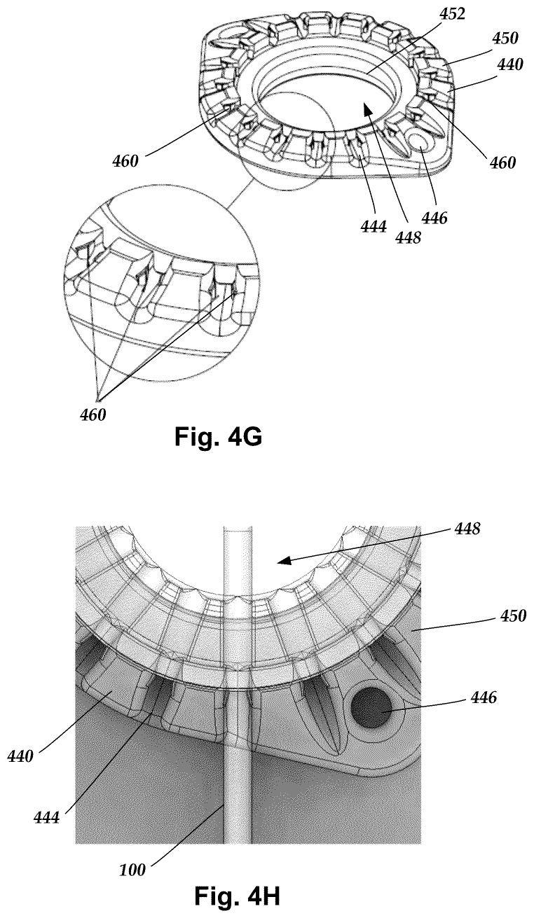

FIGS. 4A-4H illustrate a burr hole plug 445 that includes a plug base 440 and a cover 442. As illustrated in FIG. 4C, the plug base 440 can include a mount portion 450, which is mounted on the skull of the patient, and a burr hole portion 452 that is inserted into a burr hole in the skull of the patient. In at least some embodiments, the burr hole plug 445 extends no more than 4, 4.5, 5, 6, 8, or 10 mm above the skull of the patient. In at least some embodiments, the mount portion 450 of the burr hole plug 445 has a maximum width or diameter that is no more than 20, 25, 30, 35, 40, or 50 mm. In at least some embodiments, the burr hole aperture 448 or the burr hole portion 452 has a maximum width or diameter of no more than 10, 12, 14, 15, 18, 20, 25, or 30 mm.

The plug base 440 and cover 442 can be made of any suitable material including, but not limited to, rigid plastics, metals, alloys, or any other suitable biocompatible material. In at least some embodiments, if the plug base 440 or cover 442 is made of metal or alloy, the plug base may also include a non-conductive polymer disposed over all or part of the metal or alloy. For example, all or a portion of the plug base 440 can be covered by silicone or the like. In at least some embodiments, the plug base 440, cover 442, or both (or portions of one or both of these components) are made of materials including at least one of polyether ether ketone (PEEK), polycarbonate, or a combination thereof. In at least some embodiments, the cover 442 can be made partially or completely of a flexible or compressible material, such as, for example, silicone.

The plug base 440 defines one or more fastener apertures 446 through the plug base for attachment of the burr hole plug 445 to the skull 490. A fastener (not shown), such as a screw or rod, can be inserted through the fastener aperture 446 of the plug base and used to secure the plug base to the skull 490 of the patient, as illustrated in FIGS. 4E and 4F. The plug base 440 defines a burr hole aperture 448 that is placed over a burr hole formed in the skull 490 of a patient and through which the leads 100 extend through the plug base and the skull of the patient to access the brain of the patient, as illustrated in FIG. 4E. In at least some embodiments, coupling the cover 442 to the plug base 440 will resist or prevent the flow of fluid from outside the burr hole plug 445 into the burr hole aperture 448.

The plug base 440 defines multiple grooves 444 through which the lead(s) 100 can pass to exit the burr hole plug 445, as illustrated in FIGS. 4E, 4F, and 4H. In at least some embodiments, the plug base 440 includes at least one, two, three, four, six, eight, ten, twelve, sixteen, eighteen, or more (or any other suitable number of) grooves 444.

In at least some embodiments, as illustrated in FIG. 4D, the cover 442 includes a cover base 454 with one or more prongs 456 extending from the cover base and configured to engage one or more of the grooves 444 of the plug base 440 to hold the cover in engagement with the plug base. In at least some embodiments, a portion of each of the prongs 456 is complementary to a portion of each of the grooves 444 to facilitate reliable and persistent attachment of the cover 442 to the plug base 440. In at least some embodiments, the cover 442 or the prongs 456 are made partially or completely of a flexible or compressible material such as, for example, silicone to, at least in some embodiments, create a seal in or around the grooves 444 to hinder or prevent fluid flow into the burr hole aperture 448. In at least some embodiments, the prongs 456 and grooves 444 include complementary features to maintain engagement of the cover 442 to the plug base 440 absent deliberate and sufficient force applied to the cover for release from the plug base. In at least some embodiments, the prongs 456 are resilient or flexible to accommodate the portion(s) of any lead received in the grooves 444. In at least some embodiments, the cover 442 includes an aperture engagement portion 458 (FIG. 4A) that can engage an interior surface 452′ of the burr hole portion 452 to hold the cover in engagement with the plug base 440. In at least some embodiments, the prongs 456 or the aperture engagement portion 458 are resilient or flexible to accommodate the portion(s) of any lead received in the grooves 444 or extending out of the burr hole aperture 448.

As illustrated in FIGS. 4E to 4H, one or more leads 100 extend into the brain of the patient and out the burr hole aperture 448. To fasten each lead 100 within the burr hole plug 445, the lead is bent and a portion of the lead is inserted into one of the grooves 444. Any groove 444 can be selected for a particular lead 100 and the selection may be made based on the lead position, lead angle, position of the control module 102, or the like or any other suitable consideration or combination of considerations. The cover 442 is then placed over the plug base 440 to prevent the lead(s) 100 from exiting the groove(s) 444.

In can be desirable to have more secure fixation of the lead(s) 100 in the grooves 444 of the plug base 440. In at least some embodiments, it is desirable to prevent or resist sliding or lateral movement of the lead(s) 100 within the groove 444. As illustrated in FIGS. 4G and 4H, in each groove 444 of the plug base 440, one or more grips 460 that extend from at least one groove wall 462 into the groove 444 to facilitate retention of the lead 100 within the groove or to resist sliding or lateral movement of the lead 100 within the groove 444. In at least some embodiments, the grips 460 may also reduce the communication of stress applied to a lead 100 at a position exterior to the burr hole plug 445 to the portion of the lead 445 extending into the brain through the burr hole aperture 448 defined by the burr hole plug.

The grips 460 can be made of the same material or a different material from the plug base 440. In at least some embodiments, the grips 460 can be made entirely or partially of a rigid plastic material such as, for example, PEEK, polycarbonate, or the like or any combination thereof. In at least some embodiments, the grips 460 can be made entirely or partially of a flexible or compressible material such as, for example, silicone. The grips 460 can be molded or otherwise formed as part of the plug base 440 or the grips can be attached to the plug base. The grips 460 can have any suitable shape. In at least some embodiments, the grips 460 have a hemispherical or other rounded shape; a triangular (blunt or sharp) shape; a pyramidal (blunt or sharp) shape; a portion of a pentagonal, hexagonal, octagonal or any other polygonal prism; or the like or any combination thereof.

In at least some embodiments, each groove 444 includes one grip 460 extending from only one groove wall 462. Any other suitable number of grips 460 can be provided in each groove 444, such as, for example, one, two, three, four, or more grips. In at least some embodiments, each groove 444 includes one (or more) grip(s) 460 extending from each of two opposing groove walls 462, as illustrated in FIG. 4G. In at least some embodiments, grips 460 on opposing groove wall 462 can be directly opposite each other or offset from each other (for example, laterally or with respect to a height direction of the groove 444 or any combination thereof).

In at least some embodiments, the grip(s) 460 of each groove 444 can compress a portion of a lead 100 inserted into the groove, as illustrated by FIG. 4H. In at least some embodiments, the grip(s) 460 of each groove 444 only provide frictional or contact resistance to removal of a portion of the lead 100 inserted into the groove.

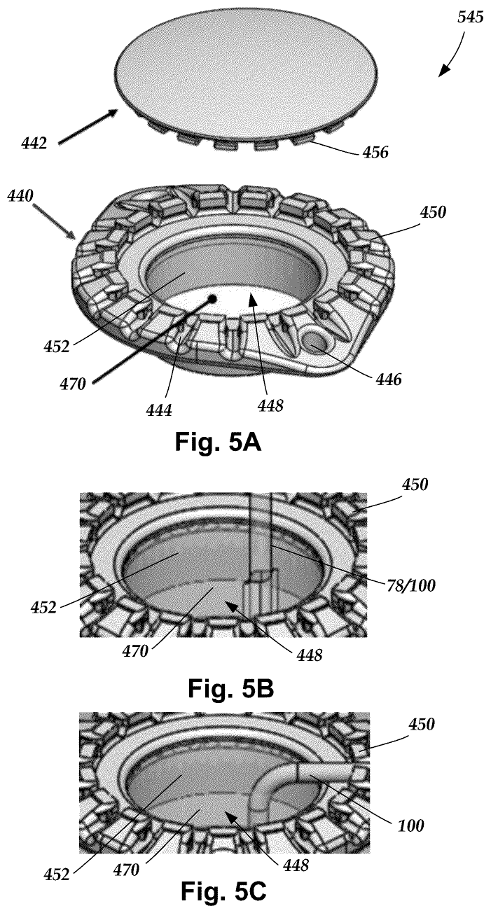

In addition to, or as an alternative to, the grips 460, another embodiment of a burr hole plug 545 includes a solid filler 470 that fills at least a portion of the burr hole aperture 448 (for example, all or part of the burr hole portion 452), as illustrated in FIGS. 5A to 5C. The burr hole plug 545 can include any combination of the components and features of burr hole plug 445 described above including, but not limited to, the plug base 440, cover 442, grooves 444, fastener apertures 446, burr hole aperture 448, mount portion 450, burr hole portion 452, and prongs 456.

The solid filler 470 is a solid, biocompatible material, such as silicone (e.g., soft silicone), a biocompatible gel, or the like, that can be pierced for insertion of a lead 100 through the plug base 440. In at least some embodiments, the lead 100 is inserted through the plug base 440 using an insertion tool 78 (FIG. 1), such as a cannula or the like, as illustrated in FIG. 5B. In at least some embodiments, the solid filler 470 is self-healing, resilient, or otherwise sufficiently flexible to fill an opening made through the solid filler or engage a lead 100 inserted through the solid filler, as illustrated in FIG. 5C. In at least some embodiments, the solid filler 470 is self-healing, resilient, or otherwise sufficiently flexible to fill a gap formed by withdrawal of an insertion tool leaving the lead inserted through the plug base.

As an example, in at least some embodiments, an insertion tool 78 (FIG. 1), such as a cannula, is inserted through the solid filler 470 in the burr hole aperture 448 and into the brain of the patient. A lead 100 is inserted through the insertion tool 78 into the brain (or inserted in conjunction with the insertion of the insertion tool) and the insertion tool is then removed. In at least some embodiments, the solid filler 470 then fills into the gap formed by removal of the insertion tool. The solid filler 470 provides a level of stabilization and securement of the lead 100 that is greater than if the solid filler 470 were absent. In at least some embodiments, the solid filler 470 facilitates maintaining the selected trajectory of the lead 100 into the brain. In at least some embodiments, the solid filler 470 resists one or more of lateral movement, longitudinal movement (e.g., sliding into or out of the brain), rotational movement, or angular movement (or any combination thereof) of the lead 100.

In at least some embodiments, the solid filler 470 fills the entire burr hole aperture 446 of the burr hole plug 545 or fills at least (or no more than) 25, 30, 33, 40, 50, 60, 66, 70, 75, 80, or 90 percent of the burr hole aperture. In at least some embodiments, the solid filler 470 fills the entire portion of the burr hole aperture 446 defined by the burr hole portion 452 (and not the mount portion 450) of the burr hole plug 545 or fills at least (or no more than) 25, 30, 33, 40, 50, 60, 66, 70, 75, 80, or 90 percent of the portion of the burr hole aperture 446 defined by the burr hole portion 452 (and not the mount portion 450) of the burr hole plug 545. In at least some embodiments, the solid filler 470 fills the entire portion of the burr hole aperture 446 that is configured to reside below an outer surface of the skull of the patient when the burr hole plug is implanted into the skull or fills at least (or no more than) 25, 30, 33, 40, 50, 60, 66, 70, 75, 80, or 90 percent of the portion of the burr hole aperture 446 that is configured to reside below an outer surface of the skull of the patient when the burr hole plug is implanted into the skull.

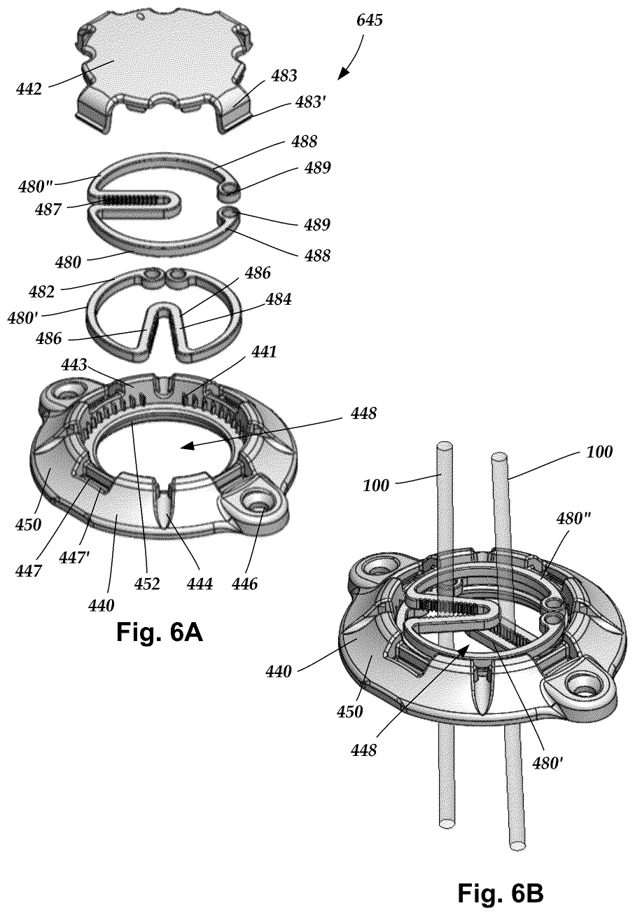

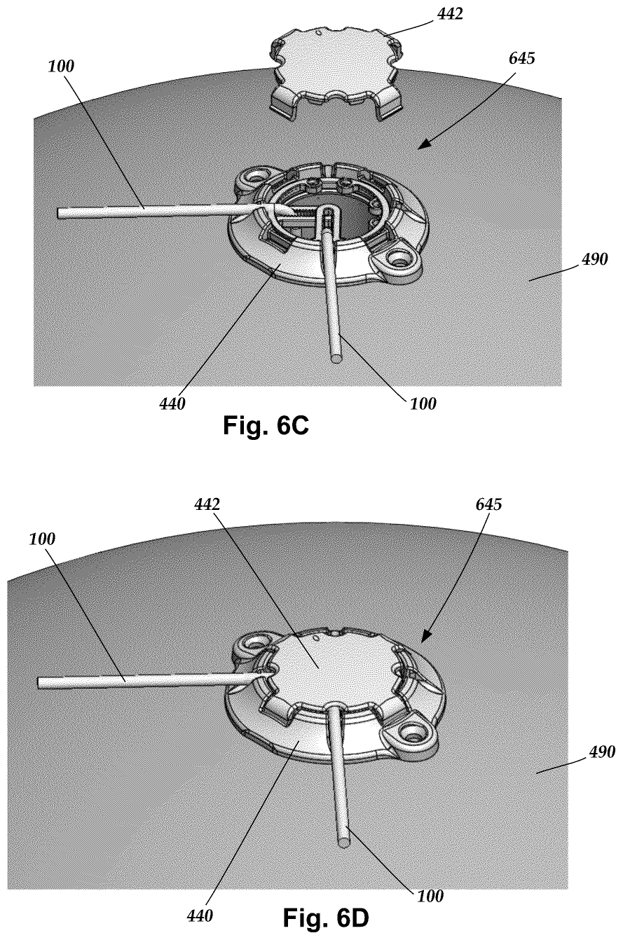

In addition to, or as an alternative to, the grips 460 or the filler 470 (or any combination thereof), another embodiment of a burr hole plug 645 includes at least one clip ring 480 that is inserted into the burr hole aperture 448 (for example, all or part of the burr hole portion 452) and receives, and clips to, a portion of a lead 100, as illustrated in FIGS. 6A to 6D. The burr hole plug 645 can include any combination of the components and features of burr hole plug 445 described above including, but not limited to, the plug base 440, cover 442, grooves 444, fastener apertures 446, burr hole aperture 448, mount portion 450, and burr hole portion 452.

A burr hole plug 645 can include any number of clip rings 480 including, but not limited to, one, two, three, four, or more clip rings. In at least some embodiments, a clip ring 480 is provided for each lead 100. In at least some embodiments, when the burr hole plug 645 includes multiple clip rings 480, the clip rings can stack within the burr hole aperture 448, as illustrated in FIG. 6B.

The clip ring 480 includes a ring portion 482 for engaging the plug base 440 and a clip portion 484 for removably receiving and holding a portion of a lead 100, as illustrated in FIGS. 6B and 6C. In at least some embodiments, the clip portion 484 includes two opposing arms 486. In at least some embodiments, the two opposing arms 486 are coupled together, as illustrated in FIG. 6A.

In at least some embodiments, the ring portion 482 has two opposing, separated, circumferential arms 488 with opposing terminal ends 489, as illustrated in FIG. 6A. In at least some embodiments, force can be applied to reduce the diameter of the clip ring 480, as illustrated by clip ring 480′ of FIG. 6A, for example, by reducing a separation distance between the opposing terminal ends 489 of the circumferential arms 488 or by increasing a separation distance between portions of the opposing arms 486 of the clip portion 484. This configuration of the clip portion 484 facilitates insertion of a portion of a lead 100 between the opposing arms 485 of the clip portion 484.

In at least some embodiments, the clip ring 480 is biased to return to a configuration with a larger diameter, as illustrated by clip ring 480″ of FIG. 6A, when the force is removed. In at least some embodiments, in this configuration, the opposing arms 486 of the clip portion 484 compress the portion of the lead 100 that is received between the opposing arms to resist or prevent sliding (e.g., longitudinal movement) or lateral movement of the lead relative to the clip ring 480.

In at least some embodiments, the opposing arms 486 include ridges 487 (e.g., teeth or other protruding features) to facilitate gripping the portion of the lead received between the opposing arms. In at least some embodiments, the plug base 440 can include ridges 441 (e.g., teeth or other protruding features) on an interior surface 443 to resist rotation of the clip ring 480 within the plug base. In at least some of these embodiments, the ring portion 482 can also include ridges (e.g., teeth or other protruding features) to engage the ridges 441 on the interior surface 443 of the plug base 440.

The clip ring 480 can be made of the same material or a different material from the plug base 440. In at least some embodiments, the clip ring 480 is made of metal, a shape memory material (for example, Nitinol™—a nickel/tin alloy—or other shape memory alloys) or a rigid plastic material such as, for example, PEEK, polycarbonate, or the like or any combination thereof.

The burr hole plug 645 includes a cover 442 that is different from the cover of burr hole plug 445. It will be understood that any of the illustrated covers 442 of FIGS. 4A to 6D can be used with any of the burr hole plugs 445, 545, 645. The cover 442 of burr hole plug 645 includes multiple clip arms 443 that extend from the cover base 454 and the plug base 440 includes corresponding receiving apertures 447 for receiving and retaining the clip arms 443 as the cover is coupled to the plug base, as illustrated in FIG. 6A. In at least some embodiments, the clip arms 443 can include a lip 443′ and the receiving apertures 447 can include an overhang 447′ that cooperate to retain engagement of the clip arms with the receiving apertures.

In at least some embodiments, the burr hole plugs 445, 545, 645 are particularly suitable for use with two, three, four, or more leads 100 inserted through the burr hole aperture 448 into the patient. A portion of each of those leads 100 is received in a different groove 444 of the plug base 440, as illustrated in FIGS. 4E, 4F, 6C, and 6D.

The above specification, examples and data provide a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. A burr hole plug, comprising:
a plug base defining a plurality of grooves and a burr hole aperture for insertion of at least one lead through the plug base, wherein each of the grooves is configured for receiving a portion of a one of the at least one lead, wherein each of the grooves is defined by at least one groove wall, wherein, for each of the grooves, the plug base further comprises at least one grip extending into the groove from the at least one groove wall to engage, and facilitate retention of, the portion of any of the at least one lead received within the groove; and
a cover configured to be disposed over, and removably coupled to, the plug base for lead retention.

2. The burr hole plug of claim 1, wherein, for at least one of the grooves, the at least one grip comprises two opposing grips extending from the at least one groove wall.

3. The burr hole plug of claim 1, wherein the grips comprise polyether ether ketone or polycarbonate.

4. The burr hole plug of claim 1, wherein the cover comprises a cover base and a plurality of prongs extending from the cover base, wherein the prongs are configured to removably engage the grooves to facilitate retention of the cover on the plug base.

5. The burr hole plug of claim 4, wherein at least a portion of each of the prongs has a shape complementary to a shape of a portion of at least one of the grooves.

6. A kit, comprising:
the burr hole plug of claim 1; and
a lead implantable within the patient through the burr hole plug, wherein the burr hole plug is configured to receive a portion of the lead within a one of the grooves.

7. The kit of claim 6, wherein the grips of the plug base are configured to compress the portion of the lead when the portion of the lead is received within the groove.

8. A burr hole plug, comprising:
a plug base defining a burr hole aperture for insertion of a lead through the plug base;
a solid filler filling a portion of the burr hole aperture, wherein the solid filler is configured for insertion of the lead through the solid filler using an insertion tool and configured to fill a gap formed by withdrawal of the insertion tool leaving the inserted through the plug base; and
a cover configured to be disposed over, and coupled to, the plug base for lead retention.

9. The burr hole plug of claim 8, wherein the solid filler comprises silicone.

10. The burr hole plug of claim 8, wherein the solid filler fills a portion of the burr hole aperture that is configured to reside below an outer surface of a skull of a patient when the burr hole plug is implanted into the skull.

11. The burr hole plug of claim 8, wherein, after the lead is inserted through the filler and the insertion tool is withdrawn, the solid filler is configured to resist at least one of lateral movement, longitudinal movement, or angular movement of a portion of the lead residing within the solid filler.

12. The burr hole plug of claim 8, wherein the plug base defines a plurality of grooves, wherein each of the grooves is configured for receiving a portion of the lead, wherein each of the grooves is defined by at least one groove wall, wherein, for each of the grooves, the plug base further comprises at least one grip extending into the groove from the at least one groove wall to engage, and facilitate retention of, the portion of the lead received within the groove.

13. A kit, comprising:
the burr hole plug of claim 8; and
the lead implantable within the patient through the solid filler of the burr hole plug.

14. A burr hole plug, comprising:
a plug base defining a burr hole aperture for insertion of a lead through the plug base;
a clip ring configured for insertion into the burr hole aperture, the clip ring comprising a ring portion and a clip portion extending radially inward from the ring portion, wherein the clip portion comprises opposing arms and, when a diameter of the ring portion is reduced by application of force, the clip portion is configured to increase a separation distance between at least some portions of the opposing arms to receive a portion of the lead between the opposing arms, wherein, when the force is removed, the ring portion is biased toward a larger diameter, complementary to a diameter of the burr hole aperture, and to engaging and compressing the portion of the lead received by the clip portion of the clip ring to retain the lead; and
a cover configured to be disposed over, and coupled to, the plug base for lead retention.

15. The burr hole plug of claim 14, wherein the opposing arms comprise ridges disposed thereon and configured to engage the portion of the lead.

16. The burr hole plug of claim 14, wherein the ring portion comprises two opposing, separated, circumferential portions extending from the clip portion.

17. The burr hole plug of claim 14, wherein the plug base comprises a plurality of ridges arranged around at least a portion of the burr hole aperture to hinder rotation of the clip ring within the burr hole aperture.

18. The burr hole plug of claim 14, wherein the burr hole plug comprises a plurality of the clip rings, wherein each of the clip rings is configured for receiving a portion of a different lead.

19. The burr hole plug of claim 14, wherein the plug base defines a plurality of grooves, wherein each of the grooves is configured for receiving a portion of the lead, wherein each of the grooves is defined by at least one groove wall, wherein, for each of the grooves, the plug base further comprises at least one grip extending into the groove from the at least one groove wall to engage, and facilitate retention of, the portion of the lead received within the groove.

20. A kit, comprising:
the burr hole plug of claim 14; and
the lead implantable within the patient through the burr hole plug.
